(19) United States
(12) Patent Application Publication
(10) Pub. No.: US 2016/0067470 A1
(43) Pub. Date: Mar. 10, 2016

(54) PARTIALLY-IMPLANTABLE PORT FOR LONG-TERM CENTRAL VENOUS CATHETER

(71) Applicant: Marcos Augusto Silva Pires e Albuquerque, Rio de Janeiro (BR)

(72) Inventor: Marcos Augusto Silva Pires e Albuquerque, Rio de Janeiro (BR)

(21) Appl. No.: 14/823,553

(22) Filed: Aug. 11, 2015

Related U.S. Application Data
(63) Continuation-in-part of application No. 11/785,714, filed on Apr. 19, 2007, now abandoned.

(30) Foreign Application Priority Data
Apr. 26, 2006 (BR) PI 0602271-5

(51) Int. Cl. A61M 39/02

(52) U.S. Cl. CPC A61M 39/0208; A61M 2039/0205

(57) ABSTRACT

A partially implantable port underneath the skin of a patient's body comprising at least one tank element, a medication outlet fixedly connected to said at least one tank element, an upright tubular element having a lower portion fixedly connected to an upper portion of said at least one tank element and having an upper portion provided with a connecting element, a blocking element being provided into said upright tubular element, said upper portion of said at least one tank element remaining outside the skin of a patient after the partially implantable port is implanted.

### CROSS-REFERENCE TO RELATED APPLICATIONS

This application is a continuation-in-part of U.S. application Ser. No. 11/785,714, filed Apr. 19, 2007, which is incorporated by reference herein in its entirety.

### FIELD OF THE INVENTION

The present invention relates to a partially implantable medical device or port which is partially implanted beneath the skin of a patient in order to allow access to the patient's vascular system so as to make possible fluid transfer between an external area of the patient's body and an internal area of the patient's body without the need to puncture the skin of the patient with a needle. The partially implantable port of the invention can be used to give intravenous fluids, medications, blood products, coagulation factors and to take blood samples, as in chemotherapy, bone marrow transplants, hemodialysis, hemotherapy, insulin injection, parenteral nutrition, etc.

### RELATED ART

It is well known in medical techniques that some medication need to be administered intravenously for long periods of time. In some medical areas, as in oncology, chemotherapy medications must be administered directly to the central venous system through devices called Long-Term Catheters which are the link between the external and intravascular environments. Said devices can be used for several months, or even years, if necessary, and typically have long idle periods between manipulations.

Currently available long-term catheters have configurations which qualify as semi-Implantable or totally-implantable catheters. These types of catheters are commonly referred to as a central venous catheter because it is inserted into a large vein leading directly into the heart.

A semi-Implantable catheter is a long one-piece hollow tube made of soft, rubber-like material called silicone, with an opening called a lumen. It is surgically inserted into one of the main blood vessels leading to the heart. The catheter exits through the patient's skin in a long external length to be connected directly into a syringe, or I.V tubing, without the use of needles. It can be provided in one lumen or multiple lumens, commonly two or three.

The BROVIAC® catheters and HICKMAN® catheters are well known trademarks of semi-Implantable catheters (Broviac J W, Cole J J, Scribner B H. A Silicone Rubber Atrial Catheter for Prolonged Parenteral Alimentation. Surg Gynecol Obstet 1973; 136:602-606. Hickman R O, Buckner C D, Cliff R A, Sauders J E, Stewart P, Thomas E D. A Modified Right Atrial Transplant Recipients. Surg Gynecol Obstet 1979; 148:871-875).

In a two or three lumens catheter the segment positioned outside the patient's body looks like two or three catheters that joint in one just before entering in the patient's body. There is no communication between the lumens of the catheter, and so different medications, fluids, blood products can be injected at the same time in the patient's body. The drugs simultaneously administered don't interact with each other before reach the blood system, which is important to keep drug's stability.

Usually a cuff, such as a Dacron® cuff, is used in a specific point around the catheter-tube to anchor the catheter under the skin. The cuff serves two purposes, to reduce the risk of dislodgement in consequence of the catheter slipping out of position and to prevent infections by stopping bacteria from entering the tunnel, traveling up to the vein and entering the blood stream.

After the long-term semi-implantable catheter is surgically inserted into the patient's body a syringe or an I.V tubing can be connected to the external end of the catheter in order to inject medication, collect blood samples, etc., whenever needed, without the use of needles.

Although long-term semi-implantable catheters are a good solution for the injection of fluids into a patient's body, mainly to the vascular system, some drawbacks are observed.

Usually they require constant dressing which can be unpleasant to patients with sensitive skin, mainly to those undergoing bone marrow transplant. Further, long-term semi-implantable catheters cause difficulties for daily showers and therefore require restricted bathing since the external catheter segment is coiled and affixed to the chest, which is a considerable inconvenience.

Furthermore the one-piece catheter configuration restricts the surgeon's technical options in accomplishing the implant, and it can be anatomically challenging for the surgeon to implant it.

Differently from the long-term semi-implantable catheter, a long-term totally-implantable catheter, as its denomination suggests, remains totally inlayed in the human body after being surgically implanted. Unlike the semi-implantable catheters it is a two-piece configuration, in which the proximal tip of the catheter-tube will be connected to the port during the implantation procedure. As it remains completely positioned under the skin it is required a needle to access its interior in order to enable drug delivery.

A long-term totally-implantable catheter, or simply a catheter tube, is used in connection with a self-sealing injection port, which consists of a tank compartment provided with a septum, usually made in silicone. The self-sealing injection port and the catheter's tube form a totally-implantable venous access system (TIVAS). The port and the catheter's tube are connected and inserted entirely under the skin, passing through surgical incisions.

In an abridged manner, the implantation of a TIVAS starts with a deep venous puncture according to Selding method (Seldinger S I. “Catheter replacement of the needle in percutaneous arteriography: a new technique”. Acta radiologica 1953; 39 (5): 368-76. doi:10.3109/000169253091367221.

In this well-known procedure a deep vein is punctured and a temporary guidewire is positioned inside the vein. Afterwards, a small incision is made few centimeters below the puncture area and a pocket under the patient's skin is performed to lodge the totally-implantable port.

Next, the port and the catheter-tube are attached together and the catheter-tube tunneled under the skin to reach the venous-puncture area. Finally, to complete the implantation, the catheter-tube is cut distally in the length desired and inserted into the vein replacing the pre-inserted guidewire. The tip of the catheter must be positioned into the Superior Vena Cava (SVC), and after the surgeon having checked out such location the incisions are closed with dissolvable sutures.

This methodology of implantation is called anterograde-technique, since the catheter is cut distally in length immediately after the tunnelization, when the proximal tip of the catheter-tube has already been attached to the port.

However, when the central venous path is affected by obstructions or deviations commonly observed in cancer situations, a retrograde-technique is necessary to overcome such obstructions or deviations. In this retrograde methodology of implant, the catheter-tube is initially positioned into the central venous system, followed by the subcutaneous tunnelization, and lastly the catheter-tube cut proximally in length to be attached to the port (See Davanzo W J. Efficacy and Safety of a Retrograde Tunneled Hemodialysis Catheter: 6-Month Clinical Experience with the Cannon Catheter™ Chronic Hemodialysis Catheter. J Vasc Access 2005; 6:38-44).

Such retrograde approach is just possible when two-pieces device systems are used, which is the case of the totally-implantable catheter. The retrograde-technique is not possible when one-piece semi-implantable catheters are used.

After the healing of the parts of the skin in the regions where incisions were made, the totally implantable venous access system-TIVAS is ready for use. If cared for correctly, a totally implantable venous access system can be used for long term.

Although a totally implantable venous access system is also a good solution for the injection of fluids into a patient's body, there are some issues which can cause problems for its use.

A totally implantable venous access system requires the use of a special 20 gauge needle, called a Huber needle, which is designed to penetrate the septum of the injection port without cutting and removing any cores or slivers from the septum in order to prevent leakages. The Huber needle is punctured through the patient's skin and next the silicon septum to inject a fluid into the tank of the injection port.

An inconvenient for the use of needles together with totally implantable venous access systems is the need to frequently puncture the skin in a same area of an already damaged skin by cancer disease could possibly had caused ulceration and local infection. In such situation it is necessary to await recovering of the patient's skin to resume the use of the totally implantable venous access system. This can cause problems to the patient's treatment.

An example of such situation occurs in oncological patients who suffer from Graft-versus-host disease (GvHD), a disease generally associated with bone marrow transplant in that the newly transplanted donor cells attack the transplanted recipient's body. Consequently skin rash, discolored areas, skin tightening or thickening normally appear. In these situation is very difficult to frequently puncture the patients' skin to reach an injection port.
Another drawback of totally-implantable catheters is the fact that the use of a 20 gauge needle restricts the flow rate for the injection of medicines, whereby the stream of fluids passing through the 20 gauge needle can be considered a vascular system of low-flow rate stream. Consequently, some therapeutic oncological treatments, such as apheresis and photopheresis, or hemodialysis, in the field of nephrology, which require vascular system of high-flow rate streams, cannot be accomplished using 20 gauge needles.

Apheresis is an extracorporeal therapy in which blood compounds are separated using an apparatus called a cell separator. This procedure is used, for example, to collect blood stem cells of a donor, in which the blood of the donor is passed through a cell separator to collect stem-cells.

Photopheresis, which is also an extracorporeal therapy, is a cell-based immuno-modulatory therapy that involves a special machine collecting T lymphocytes from peripheral blood. These cells are treated extra corporeally with ultraviolet radiation before being reinjected in the blood system. The extracorporeal photopheresis procedure is used for managing acute or chronic graft-versus-host disease secondary to bone marrow transplants.

These medical treatments frequently used in protocols of bone marrow transplants require high-flow stream to pass through the totally implantable venous access system so as to be effective. To that end only the use of needles of higher diameters, as 16 gauge needles, or larger, can guarantee that such treatments can be accomplished accordingly. (Ständer H, Neugebauer F, Schneider S W, Luger T A, Schiller M. Extracorporeal photopheresis with permanent subcutaneous right atrial catheters. JDDG; 2007, 5:1112-1119. DOI: 10.1111/11610-0387.2007.06524.x).

Hemodialysis is also an extracorporeal therapy used for the clearance of the blood in situations in the patient's kidneys are in-renal failure. Millions of people have to face hemodialysis treatments, frequently from two or more days a week. A high-flow rate (300-500 ml/min) of blood throughout an access-device is necessary to accomplish hemodialysis, and to do so, large needles are needed.

However, large needles can cut and remove cores or slivers from the septa of totally-implantable ports, thereby enabling leakages into the patient's skin to occur, secondarily leading to infections. Hospital reports to the FDA of leakage after accessing the port with Huber needles made FDA conduct laboratory testing of Huber needles from multiple manufacturers. As a result, the tests showed that certain Huber needles produced septum cores when inserted into the septa of injection ports—(see http://www.fda.gov/MedicalDevices/Safety/AlertsandNotices/ucm198719.htm).

Moreover, needles, besides being painful to patients, especially to children, expose healthcare-workers who have to handle them to many sources of contamination, due to inadvertent needle injury. Most of the States of the U.S. had already enacted safe needle legislation in order protect healthcare-workers against contaminations due to inadvertent needle injuries. (Kasprak J (2003) Needlestick Laws. OLR Research Report. Available: http://www.cga.ct.gov/2003/olrdata/ph/rpt/2003-R-0464.htm [accessed May 29, 2003]).

So far there is not in the market devices specifically designed to be used in some sort of cancer protocols, such as the bone marrow transplants and their secondary complications, which require high fluid flow rates. Usually, currently available catheters are adapted to be used in such treatments, although they had not been developed for such use.

Therefore, there is clearly a need of venous access systems which render unnecessary the use of needles.

The patent application US2004/249361 discloses a percutaneous port and an external fluid guiding system able to connect to the partially implantable port system via a connecting head. The percutaneous port 1 comprises a main casing 2, an anchoring body 3, a supporting body 6 provided with a centering element 7, a membrane casing 10, a membrane 8 and an O-ring 17.

A medication outlet catheter tube 5 is fixedly connected to a lower central hollow portion of the supporting body 6, which is also provided with a plurality of recesses on its rim, said recesses forming passages for a removing tool intended to grip and remove the supporting body out of the main casing 2, whereby the medication outlet catheter tube 5 is also removed due to its fixedly connection to the supporting body 6.

As can be seen in the FIG. 2 of the patent application US2004/249361, the medication outlet catheter tube 5 runs initially from its fixedly connection to the supporting body 6 in a parallel orientation to an axis L of the percutaneous port 1, deflecting into and opening funnel formed in a lower hollow inferior portion of the main casing 2, which serves both to guide the deflection of the medication outlet catheter tube 5, to run to its end destination, and to protect it from pressure loads. In short words, the medication outlet catheter tube 5 makes a 90° curve into the funneled lower hollow inferior portion of the main casing 2.

The patent application US2004/249361 also discloses a fluid guiding system 20 formed by an external catheter 21 connected at a end to a connecting head 22 which is formed by a base body 23, a connecting cannula 28, which protrudes beyond the connecting elements 24 on the underside, two second connecting elements 24 and two griping elements 25.

According to the specification of the patent application US2004/249361, in order to implant the port 1 under the skin, firstly an operator cuts the catheter 5 distally in a sufficiently long segment for the specific application. Then it is pulled through the casing opening of the underside of the main casing 2 and implanted by the operator.

Next the supporting body 6 is moved into the main casing 2 to the position defined by the centering element 7, and the membrane casing 10 with the membrane 8 inserted into it is screwed into the main casing 2 until it presses against the supporting body 6 and hermetically seals its outlet for the implanted catheter 5 all round, forming a hollow space.

Although it is mentioned in the specification that an O-ring is used in the percutaneous port 1, and as in the FIG. 2 it is indicated a numeral reference 17 in a void space between an inner upper rim portion of the main casing 2 and an outer middle portion of the membrane casing 10, no O-ring is shown in the FIG. 2, and there is no mention in the specification of when and how it is assembled in the percutaneous port 1.

To guide infusion fluids into the patient's body via the percutaneous port 1 and the connected, implanted medication outlet catheter tube, or to remove body fluids in the reverse direction, the external catheter 21 is connected to the percutaneous port 1 with the aid of the connected catheter head 22.

The connection is established by inserting the connecting cannula 28 of the catheter head 22 into a passage of the membrane 8, along the longitudinal axis L. The penetrating connecting cannula 28 compresses the membrane 8 into indentations in the surface area of the membrane casing 10. Axially directly above the membrane 8, an equalization space is created. The membrane 8 surrounds the connecting cannula 28, forming a gas-tight connection on all sides during penetration of the cannula 28 and when it is connected.

The percutaneous port 1 of the patent application US2004/249361 has a number of drawbacks which render difficult its use. Firstly, it comprises a number of different elements which must be assembled together immediately before the percutaneous port 1 is supposed to be implanted underneath a patient's skin.

Previously to start the implantation of the percutaneous port 1 the surgeon must execute a number of steps which require extreme precision, such as passing the catheter 5 through the opening of the main casing 2, positioning the centering element 7 of the supporting body 6 in a proper recess in the main casing 2, inserting the membrane 8 into the membrane casing 10 and screwing the latter to the main casing 2, not to mention the need to install the O-ring 17 in a non-disclosed manner in the specification. In short words, the surgeon is required to accomplish at least five previous steps to start the real implantation of the percutaneous port 1.

It is important to mention that the tightness of the connection between the membrane casing 10 and the main casing 2 relies on three factors: (i) a threaded connection between the membrane casing 10 and the main casing 2; (ii) an O-ring 17 supposedly used in a void space between an inner upper rim portion of the main casing 2 and an outer middle portion of the membrane casing 10; and a compression between the outer upper part of the supporting body 6 and the inner lower part of the membrane 8.

Those skilled in mechanical connections know that threading connections and O-rings, if not assembled accordingly, are prone to leak. Therefore, any fail in the assemblage of the percutaneous port 1 can cause leakages. In case occurs a leakage from the device into the patient's skin it can cause an infection. And if such an infection occurs, there is the risk the human body tends to expel the implanted device, thereby causing the implantable device to become useless, which is a serious problem, in special when long term treatments are needed.

Another issue is the tightness provided by the membrane 8 when it is compressed by the upper part of the supporting body 6. Being manufactured in a resilient material, the membrane 8 would deform in consequence of the pressure exerted by the supporting body 6 as the latter is screwed into the main casing 2, and according to the specification it is expected that such a compression is enough to cause the membrane 8 to deform in such a way that it fulfills the hollow existing in its central longitudinal passage in order to provide a tight blockage.

In other words, when a pressure is exerted against the upper and lower parts of the membrane 8, it is very difficult that the central longitudinal passage of the membrane 8 closes in order to form a hermetical seal because in this case the material of the membrane 8 in the surrounding region of the passage would tend to deform or even to tear, due to the pressure.

The central longitudinal passage is intended to allow the insertion of the cannula 28 into it, and the membrane 8 clearly was designed to provide a seal when the cannula 28 is connected to the percutaneous port 1. The removal of the cannula 28 from the central passage of the membrane 8 could cause problems to the tightness of the hypothetically hermetical seal provided by the central passage of the membrane 8.

In short words, the compression exerted by the upper side of the supporting body 6 against underside of the membrane 8 would only cause a deformation in the membrane 6 to make it to operate as a packing element to make seal against the external wall of the cannula 28, when it is inserted. In case the cannula 28 is not inserted said pressure would not be enough to prevent a pressurized countercurrent of blood coming from a patient's vein.

Therefore, the membrane 8 was specifically designed for the situations in that the cannula 28 is inserted into the central passage of the membrane 8, but not when the cannula 28 is not in place. In the latter situation, and in case the catheter 5 is connected to a vein, a blood countercurrent would probably occur.

Apparently the catheter and port system of US2004/249361 was designed to be used in peritoneal insulin infusion, a situation in which the intracavitary pressure is zero, and in this case it is not feasible the possibility of a counter flow to occur. However, in situations in which the catheter and port system is connected to the central venous system of a patient, where the internal pressure usually varies from 8 to 15 cmH₂O, this is enough to cause a counter-flow into the catheter and port system.

If the catheter and port system is idle, and the cannula 28 is not connected to the head 22, the chances that a blood counter-flow can pass throughout the membrane 8, in view to the insufficient tightness it provides.

A further drawback of the percutaneous port 1 of the patent application US2004/249361 is that it is not possible for a surgeon to opt to make its implantation by the use of the retrograde-technique, due to the fact that the catheter 5 is fixedly connected to the lower portion of the supporting body 6, and so, it cannot be cut proximally. Besides, even if the catheter 5 were not fixedly connected to the supporting body 6, it would be very difficult, even impossible, for the surgeon to tunnel the catheter 5 towards the percutaneous port 1 and connect it to the lower connecting portion of the supporting body, in view to the fact that this connecting portion is located in an upper position of the funneled opening 4.

The surgeon would have to do a top-down approach in order to first pass the catheter 5 across the anchoring body 3 and next to connect manually its proximal tip to the supporting body 6 inside the main casing 2, making an upright 90° turn, which technically is highly unlikely

A furthermore drawback is that the percutaneous port 1 is not provided with a tank. In some situations it is necessary that the port is provided with a tank, such as:
a tank would allow the collection of samples of residual fluids remaining into it, when medicines are not being injected, thereby enabling to check the existence of bacterial colonies which could cause sepsis in the patient;
a higher volume of fluid remaining inside a tank would be better to treat internal contamination of the device by accomplishing protocols of antibiotic lock-therapy. The rescue of the integrity of a contaminated port device through this protocol is nowadays reported by many authors. (see Kim E Y, Saunders P, Yousefzadeh N. Usefulness of Anti-Infective Lock Solutions for Catheter-Related Bloodstream Infections Mt Sinai J Med 2010; 77:549-558. DOI: 10.1002/msj.20213);
a tank is also a way to allow sedimentation of intraluminal formed micro-thrombus in the bottom of it, instead of repeatedly going to the bloodstream, thereby causing thrombus embolism. Sometimes, when totally-implantable devices are taken out of patients after long-term use, a sheet of clot can be seen in the bottom of the tank.

An yet further drawback of the percutaneous port 1 of the patent application US2004/249361 is related to the possibility that an inflammatory process can occur in the parts of the skin which encircles the upper part of the main casing 2 which protrudes above the surface of the patient's skin, after the percutaneous port 1 had been implanted, in view to the large external diameter of the main casing 2.

This problem is more serious in cases in which the percutaneous port were implanted underneath the skin of patients suffering from Graft-versus-host disease (GvHD), having a severely ill skin, thereby causing difficulties for the patient's skin to heal, especially when such a large diameter piece is partially implanted into it. And it would be worse if the internal passageway of the percutaneous port 1 were sized to allow a high fluid flow rate to pass through it. In this case it would be necessary to increase the external diameter of the percutaneous port 1, and consequently of the main casing 2, which would increase even more the problems related to local infection, and secondly, rejection.

The major drawback of the percutaneous port 1 of the patent application US2004/249361 is its technical concept, relying on a number of concentric parts axially connected between them. This causes the external diameter of the port to be very thick, although the useful internal diameter of its internal passageway in relatively thin.

The relationship between the external diameter of the upper part of the main casing 2, which protrudes above the surface of the patient's skin, and the useful internal diameter of the passageway is very high, as can be seen in the FIG. 2 of the patent application US2004/249361. By watching the Figure it can be inferred that this relationship is at least of 8, meaning that the external diameter of the main casing 2 of the percutaneous port 1 is 8 times more than the useful internal diameter of its passageway.

And even if it would made an attempt to design a thinner percutaneous port 1, yet the resultant device would be thick, due to this technical concept, as such attempt would be limited by the need to have a minimum mass of material to give strength to the screwed connections.

In view of the above analysis, it is clear that the percutaneous port 1 of the patent application US2004/249361 fails to solve the problems encountered in the implantation of partially-implantable venous access systems and in its use.

### SUMMARY OF THE INVENTION

It is an object of the present invention the provision of a partially implantable port which is partially implanted beneath the skin of a patient that precludes the use of needles.

It is a further object of the present invention the provision of a partially implantable port having an upright tubular element whose upper portion remains outside of the skin of a patient.

It is another object of the invention the provision of a partially implantable port having an upright tubular element in which the relationship between its external diameter and its internal diameter is close to 1.

It is yet another object of the invention the provision of a partially implantable port which enables a high flow rate to pass through its internal passageway.

It is still another object of the invention to provide a partially implantable port which enables a high flow rate to pass through its internal passageway without turbulence.

The partially implantable port can be embodied in single or multiple lumens, and can be used in a number of medical treatments which require access to the interior of the body of a patient, mainly the central venous system.

The invention will be fully understood by reading the following detailed description in conjunction with the accompanying drawings.

### BRIEF DESCRIPTION OF THE DRAWINGS

FIG. 1 depicts a perspective view partially in cross section of a first embodiment of the partially implantable port for long-term central venous catheter object of the present invention.

FIG. 2 depicts a partial cross sectional view of the partially implantable port for long-term central venous catheter depicted in FIG. 1.

FIG. 3 depicts a partial view of a human body, showing some internal organs, to illustrate a partially implantable port for long-term central venous catheter implanted to the human body.

FIG. 4 depicts a perspective view of a second embodiment of the partially implantable port for long-term central venous catheter object of the present invention.

FIG. 5 depicts a partial cross sectional view showing internal components of the partially implantable port for long-term central venous catheter depicted in FIG. 4.

FIG. 6 de depicts a further partial cross sectional view showing internal components of the partially implantable port for long-term central venous catheter depicted in FIG. 5 showing an IV tubing inserted into an upright tubular element of the partially implantable port.

FIG. 7 depicts an exploded view of the second embodiment of the partially implantable port for long-term central venous catheter object of the present invention.

### DETAILED DESCRIPTION OF THE INVENTION

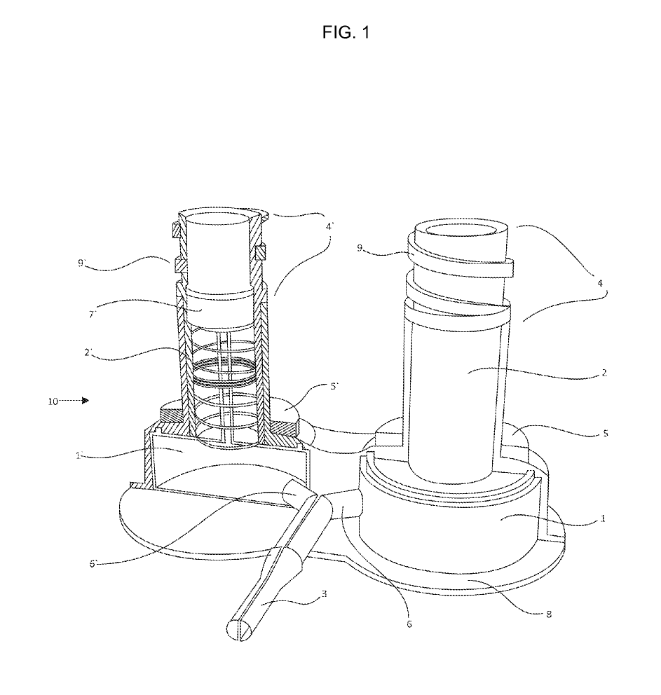

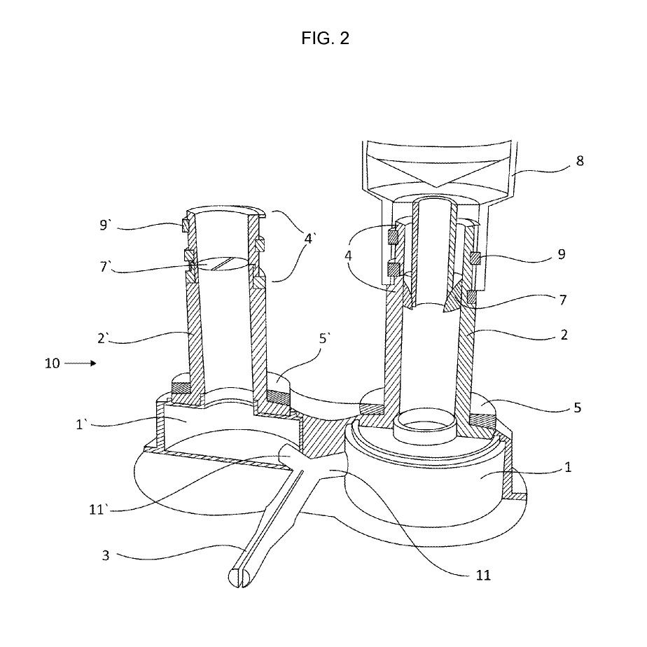

FIG. 1 depicts a perspective view of a first embodiment of a partially implantable port 10 for long-term central venous catheter object of the present invention. FIG. 2 depicts a cross sectional view of the partially implantable port 10 depicted in FIG. 1, showing some of its internal components.

The partially implantable port 10 comprises first and second tank elements 1, 1′ assembled sidelong each other. First and second tank outlets 11, 11′, respectively connected an one end to the first and second tank elements 1, 1′, unite in their middle to distal ends to form a medication outlet 3, the latter serving as a connection to a dual-lumen catheter to be connected to the partially implantable port 10.

In this embodiment the first and second tank outlets 11, 11′ have the shape of semicircular tubings. In case the partially implantable port 10 is provided with only one tank element, then only one tank outlet is provided and consequently the medication outlet and the tank outlet will be the same component.

Innovative first and second upright tubular elements 2, 2′, shaped like towers, raise upwards from and are respectively and fixedly connected to an upper portion of the first and second tank elements 1, 1′.

First and second band element 5, 5′ respectively encircle and adhere to the first and second upright tubular elements 2, 2′ and to the first and second tank elements 1, 1′ in the region where the first and second upright tubular elements 2, 2′ are fixedly connected to the first and second tank elements 1, 1′, respectively.

The first and second band element 5, 5′ are designed to block the external environment from the inner part of the human body and thus to protect the patient against infections and to prevent the patient's body to reject the partially implantable port 10 after being implanted underneath the patient's skin. The preferred material for manufacturing the first and second band element 5, 5′ is polyethylene terephthalate.

The external surface of the partially implantable port 10 is coated with a medical grade silicone rubber, except for in the first and second upper portions 4, 4′ of the first and second upright tubular elements 2, 2′, respectively. First and second connecting elements 9, 9′, such as Luer-Lock connectors, are respectively provided in these first and second upper portions 4, 4′, serving to connect to injecting devices, like I.V. tubings. FIG. 2 depicts an I.V. tubing 8 connected to the first connecting element 9 of the first upright tubular element 2.

The partially implantable port 10 must be a closed system, and to the end first and second blocking elements 7, 7′ are assembled into the first and second upright tubular elements 2, 2′, respectively.

It should be noted that only the second blocking element 7′ is shown in FIG. 1, in view to the fact that the second upright tubular element 2′ is shown in cross section, showing into it the second blocking element 7′. As the first upright tubular element 2 is shown in its entirety in FIG. 1, therefore the first blocking element 7 cannot be seen in the Figure, as it is assembled into the first upright tubular element 2.

The first and second blocking elements 7, 7′ can be of any kind of anti-return valve suitable to guarantee that no counter flow can pass from the first and second tank elements 1, 1′ through the first and second upright tubular elements 2, 2′, respectively.

For the sake of exemplification only, two different types of blocking valves are depicted in the Figures. A first type, designated as “active”, which makes use of a coiled spring, is shown in FIG. 1, into the second upright tubular element 2′, in a closed position.

A second type of valve, designated as “passive” is made of an appropriate silicone. Two of these valves are shown in FIG. 2 into the first and second upright tubular elements 2, 2′. Notice that the valve inside the second upright tubular element 2′ is in a closed position, and the valve inside the first upright tubular element 2 is in an open position, due to the I.V. tubing 8 being connected to the first connecting element 9 of the first upright tubular element 2.

Both exemplary valves are assembled into the first and second upright tubular elements 2, 2′ in order to block any communication from the patient's vascular system with the external environment during idle times.

The embodiment of the partially implantable port 10 depicted in FIGS. 1, 2 is a double lumen port, able to be used with dual-lumen catheters, in which two lumen ports are assembled sidelong each other to form a one piece device, thereby allowing the injection in the patient's body of two different medications at the same time without being mixed up inside the device.

It is important to observe that although the FIGS. 1 and 2 depict an embodiment of the invention comprising a two lumen port, the invention is not limited to this embodiment. For example, it is possible to embody the invention as a single lumen port, intend to be used with a single-lumen catheter, therefore having a single inventive upright tubular element. In this case the tank outlet of the single tank element would form the medication outlet of the single lumen partially implantable port.

The implantation of the partially implantable port 10 into a patient's body is made in a similar manner as the implantation of a totally implantable port. The surgeon makes a small incision in the patient's skin and next a pocket under the patient's skin is performed to lodge the partially implantable port 10, except for the first and second upper portions 4, 4′ of the first and second upright tubular elements 2, 2′, which remain outside the patient's skin.

The surgeon can opt to use the anterograde-technique or the retrograde-technique to connect a totally implantable catheter to the medication outlet 3 of the partially implantable port 10, depending on the situation.

After the healing of the part of the skin where the incision was made to implant the partially implantable port 10, it is ready for use. If cared for correctly, the partially implantable port 10 can be used for long term.

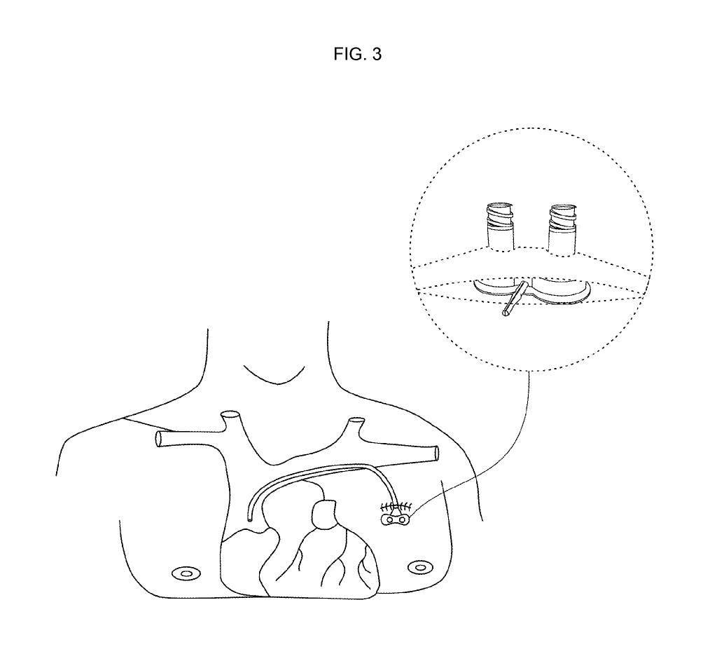

FIG. 3 depicts a partial view of a human body, showing some internal organs, to illustrate a partially implantable port 10 for long-term central venous catheter implanted to the human body.

The operation of a partially implantable port 10 after being implanted underneath a patient's skin is made by connecting a syringe or I.V. tubing directly to the inlet at the top the desired upright tubular element. When this occurs, the blocking element inside the upright tubular element is pushed downwards and opens whereby fluid can be injected into the partially implantable port 10. This operation does not require the use of needles.

The inventive provision of the upright tubular elements to the partially implantable port 10, in order to provide an “almost” totally-implantable port, wherein only an upper part of the upright tubular elements remains above the skin, allows it to be manipulated without needles, unlike the totally-implanted ports, where the whole device is under the skin and requires a needle to be accessed.

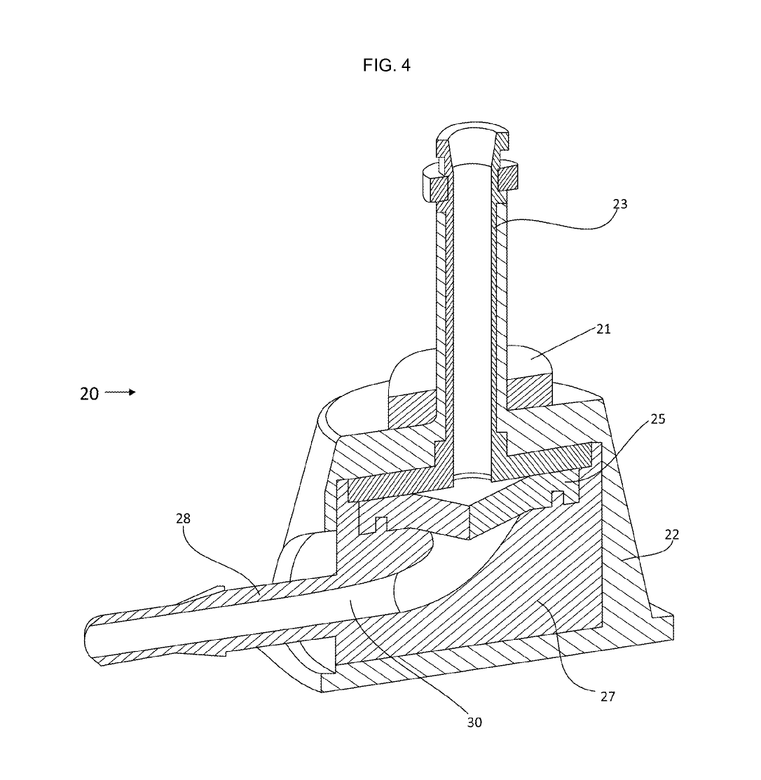

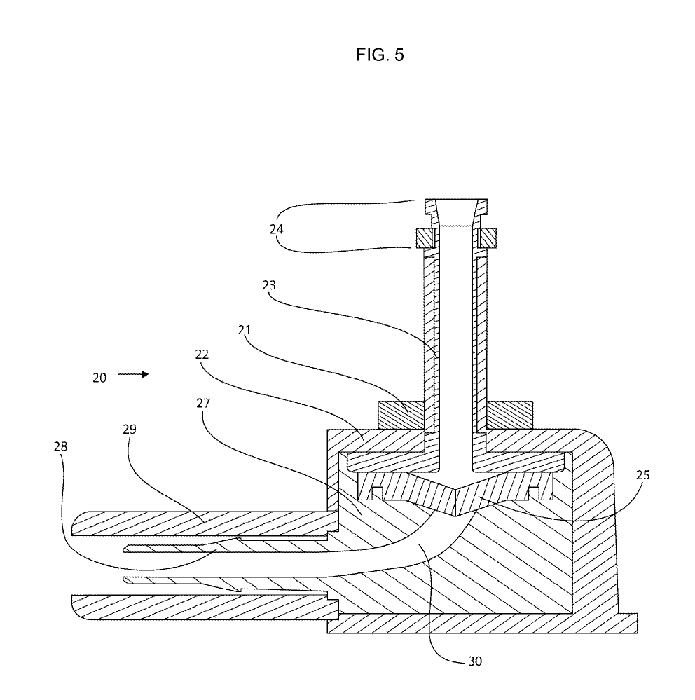

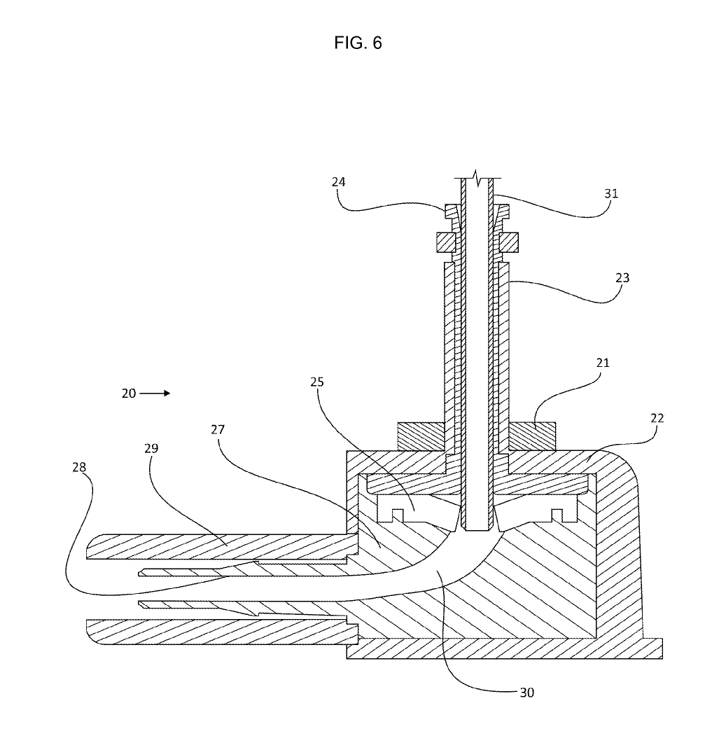

FIG. 4 depicts a perspective view in partially cross section of a second embodiment of a partially implantable port 20 for long-term central venous catheter object of the present invention. FIGS. 5 and 6 depicts cross sectional views of the partially implantable port 20 depicted in FIG. 4, showing some of its internal components.

It is important to mention that although the FIGS. 4, 5 and 6 depict an embodiment of the partially implantable port 20 comprising a single lumen port, intended to be used with single-lumen catheters, the invention is not limited to this embodiment.

For example, it is possible to embody the invention as a double lumen port, intend to be used with a double-lumen catheter, in which two lumen ports are assembled sidelong each other to form a one piece device, each lumen port having an inventive upright tubular element, thereby allowing the injection in the patient's body of two different medications at the same time without being mixed up inside the device.

In this case, likewise in the partially implantable port 10 shown in FIGS. 1 and 2, tank outlets are fixedly connected to each of the tank elements, and the tank outlets unite in their middle to distal ends to form the medication outlet.

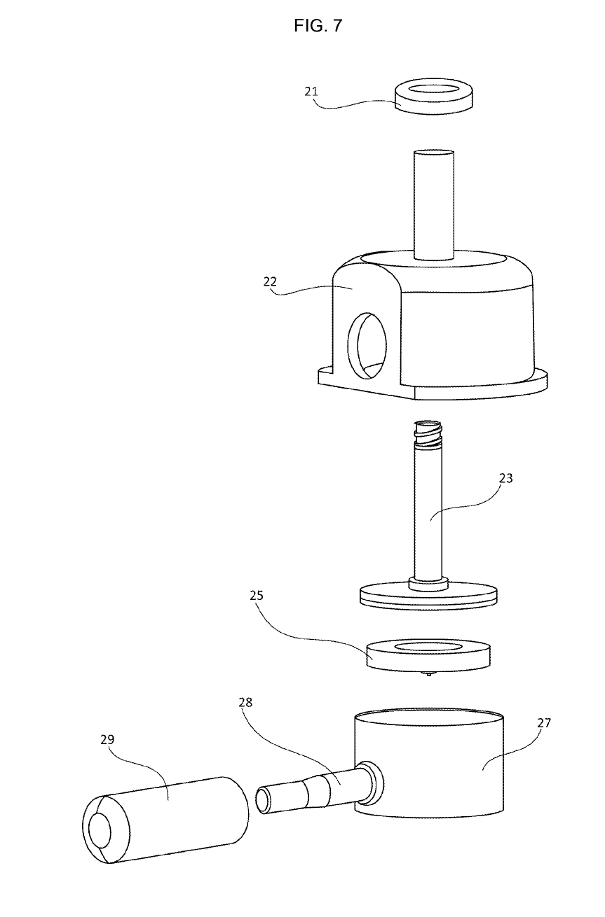

FIG. 7 depicts an exploded view of the partially implantable port 20, where is possible to see any of its components separately.

The partially implantable port comprises a tank element 27 provided with a tank inlet 30. A medication outlet 28 is fixedly connected to the tank element 27, which serves to connect to a catheter. A fixing-ring 29 encircles the medication outlet 28 in order to tighten the proximal segment of the catheter, having a suitable length to avoid fluid leakage.

A fixing-ring to firmly connect a catheter to a medication outlet can also be used in the implantable catheter 10 of FIGS. 1 and 2, as it is well known in the art.

An upright tubular element 23 is fixedly connected to an upper part of the tank element 27, forming the body of the partially implantable port 20. The upright tubular element 23 is provided with an upper connecting part 24 in its uppermost end.

A coating 22 covers the tank element 27 and the upright tubular element 23. Preferably the material of the coating 22 is silicone rubber. The upper connecting part 24 of the upright tubular element 23 is not covered by the coating 22.

A band element 21 encircles the region of the lower portion of the upright tubular element 23. The band element 21 is designed to block the external environment from the inner part of the human body and thus to protect the patient against infections and to prevent the patient's body to reject the partially implantable port 20 after being implanted underneath the patient's skin. The preferred material for manufacturing the band element 21 is polyethylene terephthalate.

A blocking element 25 is provided into the tank element 27, located between the end of the internal passageway of the upright tubular element 23 and the tank inlet 30 of the tank element 27. In the Figures the blocking element 25 is a passive anti-return valve, but the invention is not limited to this kind of blocking element. Any suitable anti-return valve can alternatively be used.

The tank element 27 is formed by an elongated tubular segment extending from the end of the tank inlet 30 to the region where the medication outlet 28 is fixedly connected to it.

If it is needed a tank element of higher capacity, it is possible to smoothly enlarge the internal diameter of this elongated tubular segment immediately after the end of the tank inlet 30. In this case it is also necessary to smoothly decrease the internal diameter of the elongated tubular segment nearby the region where the medication outlet 28 in fixedly connected to the tank element 27, in order to avoid the formation of turbulences in this region of transition of diameters.

The tank inlet 30 is a 90° long radius curve destined to provide a smooth deviation between right angled passageways, with the purpose to create conditions for a flux passing into such passageways be not turbulent.

Some medical treatments, such as hemodialysis, require that the flow rate passing throughout the port has to be as higher as possible, around 500 ml/min. In such a high rate flow it is important to avoid turbulences in the flow, which could cause hemolysis of the red blood cells, and in the following, jaundice.

The use of a tank inlet 30 having a 90° long radius curve in the partially implantable port 20 precludes turbulences from occurring into it.

FIG. 6 shows an I.V. tubing or cannula 31 inserted into the upright tubular element 23. It is provided with a means to connect to the upper connecting part 24 of the upright tubular element 23 (not shown in the drawing) in a manner well known in the art.

It can be seen that the end of the cannula 31 is above the beginning of upper part of the tank inlet 30, after having passed through the blocking element 25. In this situation the stream of downward fluids can flow with no turbulence.

For the implantation of the partially implantable port 20 into a patient's body the surgeon makes a small incision in the patient's skin and next a pocket under the patient's skin is performed to lodge the partially implantable port 20, except for the upper connecting part 24 of the upright tubular element 23, which remains outside the patient's skin.

The surgeon can opt to use the anterograde-technique or the retrograde-technique to connect a totally implantable catheter to the medication outlet 28 of the partially implantable port 20, depending on the situation.

After the healing of the part of the skin where the incision was made to implant the partially implantable port 20, it is ready for use. If cared for correctly, the partially implantable port 20 can be used for long term.

A major advantage for the use of the of the present invention is related to the fact that that the partially implantable ports 10 and 20 are provided with inventive upright tubular elements (1, 1′ and 23), which although being able to allow high flow rates of fluids to pass through them are very thin.

The external diameter of the upright tubular element is just slightly higher than the internal diameter, the increase being only the thickness of the walls of the upright tubular element. In this case the relationship between the external diameter and the internal diameter is close to 1.

Therefore, only a substantially small region of the patient's skin encircles the upper parts of upright tubular elements (1, 1′ and 23) that remain outside of the skin of the patient, and consequently the risks for an infection to occur substantially decrease. This feature is particularly important in situations that the patient suffers from Graff-versus-Host disease.

In some embodiments a tank inlet 30 comprising a 90° long radius curve in order to preclude turbulences from occurring into the partially implantable port 20.

The partially implantable ports of the invention although being able to allow high flow rates of fluids to pass through them need not to be embodied in bigger dimensions than the currently available totally implantable ports.

The invention was described herewith with regard to some preferred embodiments, but it is not limited to such embodiments. Rather, it should be considered with regard to its broader disclosure.

Modifications can be made in the invention without departing from its inventive concept, the provision of upright tubular elements of small diameters, which have its upper part located outside the patient's skin.

Although specific terms may be used herein, they were used only in a generic and descriptive form and not for the purpose of limitation of the invention.

### LIST OF COMPONENTS

1—first tank element
1′—second tank element
2—first upright tubular element
2′—second upright tubular element
3—medication outlet
4—first upper portion (of the first upright tubular element 2)
4′—second upper portion (of the second upright tubular element 2)
5—first band element
5′—second band element
6—coiled spring
7—first blocking element
7′—second blocking element
8—I.V. tubing
9—first connecting element (of the first upright tubular element 2)
9′—second connecting element (of the second upright tubular element 2)
10—partially implantable port
11—first tank outlet
11′—second tank outlet
20—partially implantable port
21—band element
22—coating
23—upright tubular element.
24—upper connecting part
25—blocking element
27—tank element
28—medication outlet
29—fixing-ring
30—tank inlet
31—I.V. tubing or cannula

1. A partially implantable port underneath the skin of a patient's body comprising:
at least one tank element;
a medication outlet fixedly connected to said at least one tank element; and
an upright tubular element having a lower portion fixedly connected to an upper part of said at least one tank element and having an upper portion provided with a connecting element.

2. A partially implantable port according to claim 1, wherein it is coated with a rubbery element, except for in an upper portion of said upright tubular element.

3. A partially implantable port according to claim 2, wherein a band element encircles said upright tubular element in a region of a lower portion of the upright tubular element located immediately above to the region where the upright tubular element is fixedly connected to said at least one tank element.

4. A partially implantable port according to claim 3, wherein a blocking element is provided into said upright tubular element.

5. A partially implantable port according to claim 4, wherein said blocking element comprises an anti-return valve.

6. A partially implantable port according to claim 5, wherein said at least one tank element comprises first and second tank elements which are fixedly assembled sidelong each other, said first and second tank elements being connected to respective first and second upright tubular elements.

7. A partially implantable port according to claim 6, wherein first and second tank outlets are fixedly connected to respective first and second tank elements, said first and second tank outlets uniting in their middle to distal ends to form said medication outlet.

8. A partially implantable port according to claim 7, wherein a fixing-ring encircles the medication outlet.

9. A partially implantable port according to claim 1, wherein said at least one tank element comprises one tank element provided with a tank outlet which forms said medication outlet.

10. A partially implantable port according to claim 9, wherein it is coated with a rubbery element, except for in an upper portion of said upright tubular element.

11. A partially implantable port according to claim 10, wherein a band element encircles said upright tubular element in a region of a lower portion of the upright tubular element located immediately above to the region where the upright tubular element is fixedly connected to said at least one tank element.

12. A partially implantable port according to claim 11, wherein a blocking element is provided in an upper portion of the tank element in a region immediately below the region where the tank element is fixedly connected to an upper part of said upright tubular element.

13. A partially implantable port according to claim 12, wherein said blocking element comprises an anti-return valve.

14. A partially implantable port according to claim 13, wherein a fixing-ring encircles the medication outlet.

15. A method to implant a partially implantable port into a living body, said partially implantable port comprising:
at least one tank element;
an upright tubular element having a lower portion fixedly connected to an upper portion of said at least one tank element and having an upper portion provided with a connecting part;
a medication outlet fixedly connected to said at least one tank element;
the method comprising the steps of:
making an incision in the skin of the living body;
making a pocket under the skin of the living body;
lodging the partially implantable port into the pocket, except for an upper portion of said upright tubular element, which remains outside the patient's skin.
